(19) United States
(12) Patent Application Publication
(10) Pub. No.: US 2026/0021277 A1
(43) Pub. Date: Jan. 22, 2026

(54) CATHETER CONFIGURED TO PREFERENTIALLY CURVE

(71) Applicant: Medtronic Ireland Manufacturing Unlimited Company, Dublin (IE)

(72) Inventors: Stefan S. Tunev, Santa Rosa, CA (US); Brian J. Kelly, Oranmore (IE); Daniel M. King, Galway (IE)

(21) Appl. No.: 18/998,836

(22) PCT Filed: Jul. 18, 2023
(86) PCT No.: PCT/EP2023/069946

§ 371 Date: Jan. 27, 2025

Related U.S. Application Data
(60) Provisional application No. 63/369,736, filed on Jul. 28, 2022.

(51) Int. Cl. A61M 25/01; A61M 25/00

(52) U.S. Cl. CPC A61M 25/0116; A61M 25/0023; A61M 25/005; A61M 25/0141

(57) ABSTRACT

A catheter including a catheter body (108) defining a catheter lumen, an expandable member (111) connected to the catheter body, and an injection tube (214, 224) disposed on an outer surface of the expandable member (111). The injection tube (214, 224) defines an injection lumen and an injection port (228) in fluid communication with the injection lumen. The catheter body (108) is configured to preferentially curve in a first direction over a second direction when the catheter is positioned in vasculature of a patient.

### TECHNICAL FIELD

The present technology is related to catheters.

### BACKGROUND

Catheters including one or more therapy elements have been proposed for use in various medical procedures, including neuromodulation procedures. For example, some catheters include an energy delivery element, a fluid delivery element, a cryogenic element, or other therapeutic elements.

### SUMMARY

The present disclosure describes catheters that include a catheter body configured to preferentially curve in a first direction over a second direction. In some examples, the catheter includes a therapy delivery element and the preferential curvature of the catheter body enables the catheter to position the therapy delivery element in a predetermined orientation within vasculature of a patient, e.g., to facilitate delivery of neuromodulation therapy (e.g., radiofrequency (RF), ultrasound, or microwave energy, a chemical agent, a thermal therapy, such as cryotherapy) in particular direction within the vasculature. For example, in some examples, the catheter includes one or more injection tubes each defining one or more respective injection ports, and the preferential curvature of the catheter body enables the catheter to position the one or more injection ports in a predetermined orientation within vasculature of a patient, e.g., to facilitate delivery of a therapeutic agent in particular direction within the vasculature. In some examples, the catheter includes an expandable member connected to the catheter body and the one or more injection tubes are disposed on an outer surface of the expandable member.

In some examples, the catheter may include a self-orienting member configured to preferentially curve the catheter body in the first direction over the second direction. The self-orienting member may be disposed within the catheter lumen or may be a part of the catheter body.

The configuration of the catheters described in this disclosure may improve the navigation and/or orientation of catheters within vasculature of the patient by ensuring that a distal portion of the catheter is oriented in a predetermined direction as the distal portion of the catheter navigates around a curve in the vasculature. In some examples, the preferential curvature of the catheter may facilitate delivery of therapy towards or away from particular anatomical features of the patient.

In some examples, the catheters described herein may be useful for neuromodulation within a blood vessel or a body lumen or cavity other than a vessel, for extravascular neuromodulation, for non-renal-nerve neuromodulation, and/or for use in therapies other than neuromodulation.

In some examples, the disclosure describes a catheter comprising: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to preferentially curve in a first position over a second direction when the catheter is positioned in a curvature in vasculature of a patient.

In some examples, the disclosure describes a catheter comprising: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to self-orient the injection port in a pre-determined orientation relative to an anatomical feature of a patient when the catheter is positioned in vasculature of the patient.

In some examples, the disclosure describes a method of delivering therapy to tissue of a patient, the method comprising: navigating a catheter through vasculature of the patient to a target treatment site, wherein the catheter comprises: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to preferentially curve in a first direction over a second direction when the catheter is positioned in the vasculature; expanding the expandable member of the catheter from a collapsed configuration to an expanded configuration; and delivering therapy to the target treatment site via the injection port.

In some examples, the disclosure describes a catheter comprising: a catheter body; and a neuromodulation element disposed on a distal portion of the catheter body, wherein the catheter body is configured to preferentially curve in a first direction over a second direction when the catheter is positioned in a curvature in vasculature of a patient.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example catheter that includes an expandable member and one or more injection tubes each defining one or more injection ports.

FIG. 2A is a conceptual diagram illustrating a cross-sectional view of a proximal portion of the catheter of FIG. 1, the cross-section being taken along line A-A in FIG. 1.

FIG. 2B is a conceptual diagram illustrating a cross-sectional view of a distal portion of the catheter of FIG. 1, the cross-section being taken along line A-A in FIG. 1.

FIG. 3 is a conceptual diagram illustrating a cross-sectional view of the example distal portion of the catheter of FIG. 2B, the cross-section being taken along line B-B in FIG. 2.

FIG. 4A is a conceptual diagram illustrating an example self-orienting member of the catheter of FIGS. 2A and 2B.

FIG. 4B is a conceptual diagram illustrating a cross-sectional view of the example self-orienting member of FIG. 4A, the cross-section being taken along line C-C in FIG. 4A.

FIG. 4C is a conceptual diagram illustrating a cross-sectional view of the example self-orienting member of FIG. 4A, the cross-section being taken along line D-D in FIG. 4A.

FIG. 4D is a conceptual diagram illustrating a cross-sectional view of the example self-orienting member of FIG. 4A, the cross-section being taken along line E-E in FIG. 4A.

FIG. 4E is a conceptual diagram illustrating a cross-sectional view of the example self-orienting member of FIG. 4A, the cross-section being taken along line F-F in FIG. 4A.

FIG. 4F is a conceptual diagram illustrating another cross-sectional view of the example self-orienting member of FIG. 4A, taken along line F-F in FIG. 4A.

FIG. 5A is a conceptual diagram illustrating a cross-sectional view of another example distal portion of the catheter of FIG. 1, the cross-section being taken along line A-A in FIG. 1.

FIG. 5B is a conceptual diagram illustrating a cross-sectional view of an example of the distal portion of the catheter of FIG. 5A, the cross-section being taken along line G-G in FIG. 5A.

FIG. 5C is a conceptual diagram illustrating a cross-sectional view of another example of the distal portion of the catheter of FIG. 5A, the cross-section being taken along line G-G in FIG. 5A.

FIG. 6 is a flow diagram illustrating an example method of delivering therapy to a patient using a catheter configured to preferentially curve.

FIG. 7 is a partial schematic illustration of another example catheter that includes a neuromodulation element.

FIG. 8 is a conceptual illustration of an example method of accessing a renal artery and modulating renal nerves with the catheter of FIG. 1.

FIG. 9 is a conceptual illustration of an example sympathetic nervous system (SNS) illustrating how the brain communicates with the body via the SNS.

FIG. 10 is an enlarged anatomic view of nerves innervating a left kidney to form the renal plexus surrounding the left renal artery.

FIG. 11 is an anatomic view of a human body depicting neural efferent and afferent communication between the brain and kidneys.

FIG. 12 is a conceptual view of a human body depicting neural efferent and afferent communication between the brain and kidneys.

FIG. 13 is an anatomic view of the arterial vasculature of a human.

FIG. 14 is an anatomic view of the venous vasculature of a human.

### DETAILED DESCRIPTION

Neuromodulation therapy (e.g., tissue ablation, tissue denervation, or the like) can be accomplished by delivering electrical, ultrasonic, and/or thermal energy to a target tissue site, and/or by delivering a chemical agent to a target tissue site. For example, the neuromodulation therapy can include denervation therapy, which is used to render a nerve inert, inactive, or otherwise completely or partially reduced in function, such as by ablation or lesioning of the nerve. Following denervation, there may be a reduction or even prevention of neural signal transmission along the target nerve. Denervating an overactive nerve may provide a therapeutic benefit to a patient. For example, renal denervation may mitigate symptoms associated with renal sympathetic nerve overstimulation. In some examples, neuromodulation therapy (e.g., denervation energy or chemical agents) is delivered via a catheter disposed in a blood vessel (e.g., the renal artery) proximate a target nerve. The catheter can be configured to be intravascularly positioned within a vessel or other anatomical lumen to deliver an energy or stimulus. The energy or stimulus may include, for example, at least one of a radio frequency (RF) stimulus, a thermal stimulus, a cryogenic stimulus, a microwave stimulus, an ultrasonic stimulus, or other form of energy or stimulus.

In examples described herein, a catheter includes a therapy delivery element configured to deliver neuromodulation therapy or another type of therapy to tissue of a patient. The therapy delivery element can be, for example, configured to deliver using RF or microwave energy, a chemical agent, cryotherapy, an ultrasound energy, or the like. A clinician may navigate the catheter through vasculature of a patient to reach a target treatment site. Once the clinician determines that the catheter is at the target treatment site (e.g., via one or more imaging techniques such as X-ray imaging, fluoroscopy, or the like), the clinician may deliver the therapy to the target treatment site via the therapy delivery element. In some examples, there may be one or more anatomical features (e.g., adjacent blood vessels) of the patient that are relatively close to the target treatment site to which the clinician does not wish to administer neuromodulation therapy. To minimize or even prevent administration of the neuromodulation energy or stimulus to the anatomical features, the clinician may need to rotate the catheter within the vasculature. Due to the length of the catheter, as well as the materials from which the catheter is formed, rotating a catheter may be relatively difficult and inaccurate when the catheter is positioned within vasculature and may lead to unintended therapy delivery to the one or more anatomical features. Further, for some catheters that are flexible enough to reach some locations, such as a renal artery, from a radial artery entry point, the catheter may not exhibit a good torque response.

The example catheters described in this disclosure are configured to improve the navigation and orientation of a therapy delivery element or other feature of the catheter within vasculature of the patient. The example catheters are configured to facilitate orientation of the therapy delivery element (or other feature) of the catheter by at least automatically self-orienting, such as by being configured to preferentially curve, the therapy delivery element as the catheter extends around a curvature in the vasculature. For example, based on knowledge of anatomy (e.g., direction and extent of curvature of vasculature) of a patient near a target treatment site, the catheters are configured to self-orient one or more electrodes, one or more ultrasound transducers, one or more thermally conductive surfaces of a balloon, one or more injection ports, or the like away from certain anatomical features and/or towards certain anatomical features. This may help reduce the likelihood that neuromodulation energy or other stimulus will be delivered to unintended tissue sites in the patient. The example catheters may also reduce an amount of time needed for the clinician to align and orientate the catheter within the vasculature and reduce an overall medical procedure time.

The therapy delivery element of the catheter can be configured to deliver the therapy in a particular direction (rather than, for example, 360 degrees around the catheter) using any suitable technique. For example, in the case of electrical energy, e.g., for RF ablation or microwave ablation, the therapy delivery element can include one or more directional electrodes, e.g., segmented electrodes that extend only partially around an outer perimeter of the catheter, ring electrodes that are partially electrically insulated to functionally achieve delivery of the electrical energy in particular direction, or the like. As another example, in the case of ultrasound energy, the ultrasound transducer can be configured to deliver the ultrasound energy in a particular direction that is less than 360 degrees around the catheter, or can be positioned in a balloon that is configured to direct the ultrasound energy in the particular direction. In the case of chemical ablation, as described below, the one or more injection ports can be positioned along the catheter to deliver a therapeutic agent in a particular direction that is less than 360 degrees around the catheter. As another example, in the case of cryoablation, a balloon can be used to deliver the cooling fluid to tissue of a patient and the balloon can be partially thermally insulated to facilitate delivery of the cooling fluid in a particular direction that is less than 360 degrees around the catheter.

In some examples, tissue neuromodulation (e.g., tissue ablation, tissue denervation, or the like) can be accomplished using high pressure chemical ablation. For example, a neuromodulation procedure can involve a high pressure introduction of a therapeutic agent into a wall of a blood vessel for neuromodulation of nearby nerves (e.g., sympathetic renal nerves in the case of a renal denervation procedure). Catheters may be used to perform needleless injection of the therapeutic agent at a target treatment site of a patient (e.g., as part of a neuromodulation procedure). The needleless injections may be performed by delivering a high pressure therapeutic agent through an injection tube (e.g., a catheter body lumen or a tube separate from a catheter body lumen) positioned in a blood vessel to force the therapeutic agent into and/or through a blood vessel wall. The chemical may then diffuse around the target treatment site (e.g., nerves surrounding the blood vessel). In some examples, needleless injections may be used to perform denervation procedures (e.g., renal denervation procedures).

In examples described herein, a catheter includes a therapy delivery element that includes at least one injection tube defining one or more injection ports configured to deliver a therapeutic agent to tissue of a patient and an expandable member configured to position the one or more injection ports in contact with tissue of a patient. To minimize or even prevent administration of a therapeutic agent to the anatomical features, the clinician may need to rotate the catheter within the vasculature. As noted above, rotating a catheter may be relatively difficult and inaccurate when the catheter is positioned within vasculature and may lead to unintended therapy delivery to the one or more anatomical features.

Some example catheters described in this disclosure are configured to improve the navigation and orientation of one or more injection ports of the catheter within vasculature of the patient. The example catheters are configured to facilitate orientation of the therapy delivery element of the catheter by at least automatically self-orienting, such as by being configured to preferentially curve, the one or more injection ports as the catheter extends around a curvature in the vasculature. For example, based on knowledge of anatomy of a patient near a target treatment site, the catheters may self-orient one or more injection ports away from certain anatomical features and/or towards certain anatomical features. This may help reduce the likelihood that a therapeutic agent or other fluid will be delivered to unintended tissue sites in the patient. The example catheters may also reduce an amount of time needed for the clinician to align and orientate the catheter within the vasculature and reduce an overall medical procedure time.

In some examples, the catheters described herein may be useful for neuromodulation within a blood vessel or a body lumen other than a vessel, for extravascular neuromodulation, for non-renal-nerve neuromodulation, and/or for use in therapies other than neuromodulation. In addition, although neuromodulation, and needleless fluid delivery neuromodulation in particular, is primarily referred to herein, the catheters described herein may be used for other neuromodulation modalities, as well as for medical procedures other than neuromodulation, including ablation of other targets.

While some examples catheters are described herein with respect to a delivery of a therapeutic agent (e.g., a chemical ablation agent), the description herein also applies to other modalities of neuromodulation therapy, including, but not limited to, RF ablation, ultrasound ablation, microwave ablation, cryoablation, and the like.

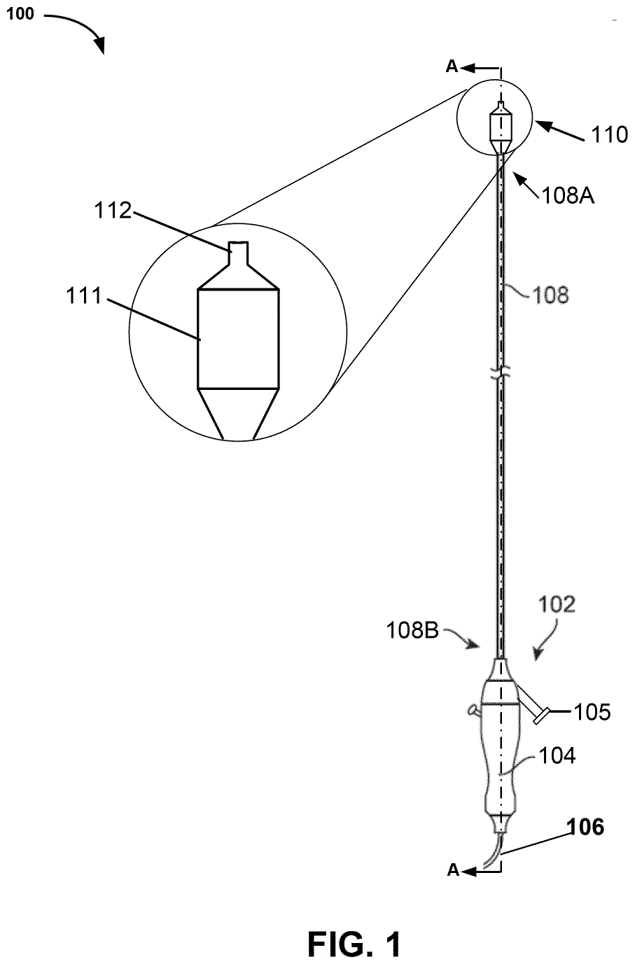

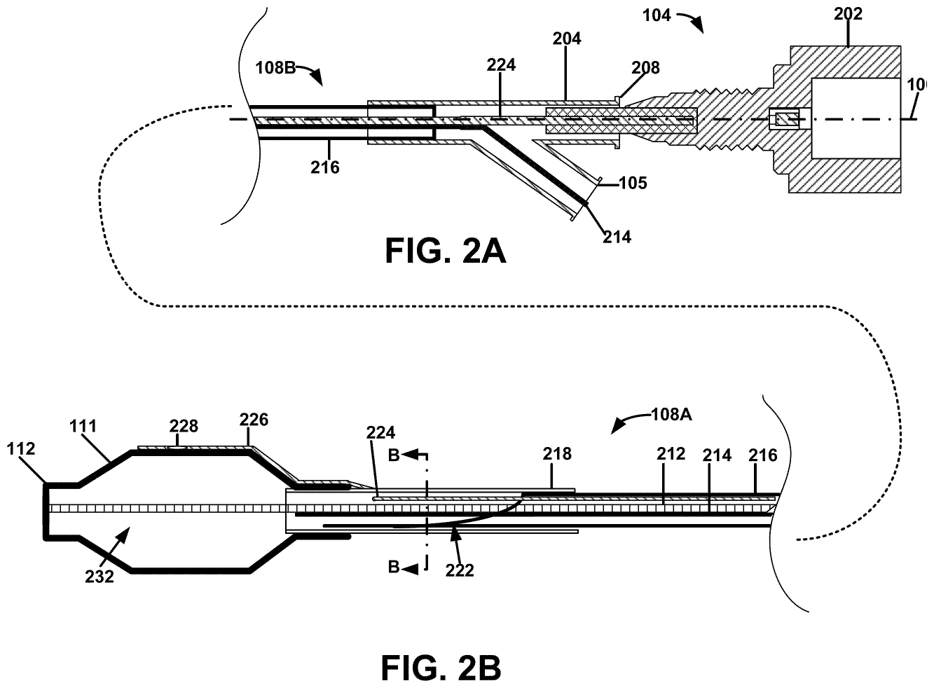

FIG. 1 is a partial schematic illustration of an example catheter 102 that includes a handle 104, an elongated body 108 attached to handle 104, and at least one therapy delivery element 110 carried by elongated body 108. Handle 104 may include a delivery port 105 configured to receive a fluid (e.g., an ablative chemical or other therapeutic agent) that is delivered via therapy delivery element 110. Therapy delivery element 110 includes expandable member 111 and one or more injection tubes (shown in FIGS. 2A and 2B) extending at least partially along an outer surface of expandable member 111. Elongated body 108 includes a distal portion 108A and a proximal portion 108B. Distal portion 108A includes a distal end 112 of catheter 102 and therapy delivery element 110. In some examples, therapy delivery element 110 may be positioned proximal to distal end 112. Catheter 102 may be a part of a medical system 100 that includes one or more other components, such as a fluid container that stores a therapeutic agent and a medical device configured to facilitate relatively high pressure delivery of the therapeutic agent from the fluid container to tissue of a patient via catheter 102.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device. For example, distal portion 108A of elongated body 108 refers to a portion of elongated body 108 at a position relatively distant from the clinician and proximal portion 108B or elongated body 108 refers to a portion of elongated body 108 at a position relatively near the clinician. In some examples, distal portion 108A is a distalmost portion of catheter 102 including a distal end of catheter 102.

Elongated body 108 has any suitable outer diameter, and the diameter can be constant along the length of elongated body 108 or may vary along the length of elongated body 108. In some examples, elongated body 108 can be 2, 3, 4, 5, 6, or 7 French or another suitable size. An outer layer of elongated body 108 may be formed from any suitable material or combination of materials, such as, but not limited to, one or more of nylon or a thermoplastic such as polyethylene terephthalate (PET), parylene, polyvinyl chloride (PVC), polyethylene, ethylene chlorotrifluoroethylene (ECTFE), or polyvinylidene fluoride (PVDF).

Therapy delivery element 110 includes one or more injection tubes (shown in FIGS. 2A and 2B) positioned along an outer surface of expandable element 111, the one or more injection tubes being configured to deliver a therapeutic agent at a target tissue site (also referred to herein as “target region”) within a patient when distal portion 108A is positioned proximate the target tissue site. The therapeutic agent may be a fluid. In some examples, while needleless fluid delivery is primarily referred to herein, in other examples, therapy delivery element 110 can be configured to deliver other types of therapy (e.g., energy, such as, but not limited to ultrasound energy, microwave energy, radiofrequency energy, electrical stimulation, or the like) in addition to a fluid and/or deliver a fluid via other structures (e.g., fluid delivery using a needle).

The injection tubes are configured to deliver a fluid from proximal portion 108B of catheter (e.g., delivery port 105) to distal portion 108A and expel a therapeutic agent out of one or more injection ports of the injection tubes at a relatively high pressure. The pressure can be selected to enable the therapeutic agent to penetrate the wall of a blood vessel of the patient, e.g., with or without a needle that penetrates into the blood vessel wall. Example pressures include, for example, 5 Megapascal (MPa) to 18 MPa (e.g., about 750 pounds per square inch (psi) to about 2500 psi), such as 10 MPa (or about 10.34 psi). The one or more injection tubes can be disposed at least partially within elongated body 108 and/or can be positioned entirely external to elongated body 108. In some examples, delivery port 105 may be proximal to or defined by handle 104.

Expandable member 111 is configured to expand radially outwards when an inflation fluid, e.g., saline, is delivered to an interior volume of expandable member 111 via an inflation lumen of catheter 102. Expandable member 111 can be, for example, a balloon (e.g., a compliant balloon). In some examples, when expanded, expandable member 111 is symmetrical (e.g., radially symmetrical and/or longitudinally symmetrical). In some examples, when expanded, expandable member 111 is asymmetrical longitudinally along longitudinal axis 106 and/or radially around longitudinal axis 106.

Although examples in which expandable member 111 includes a balloon are primarily described herein, in other examples, expandable member 111 can include a different type of expandable structure. For example, expandable member 111 can include an expandable frame, e.g., an expandable basket or stent-like structure, configured to expand radially outwards relative to elongated body 108. The expandable frame can be self-expanding (e.g., formed from one or more shape memory materials, such as nitinol) or can be configured to expand with a balloon. The expandable frame can define one or more openings through which blood may flow past expandable member 111 when expandable member 111 is expanded with a blood vessel of a patient.

As discussed in further detail with reference to FIGS. 2A and 2B, elongated body 108 includes an outer catheter body which defines an outer catheter lumen. In some examples, the outer catheter lumen is an inflation lumen in fluid communication with an inner volume of expandable member 111. A clinician may introduce an inflation fluid into and/or extract the inflation fluid from the inner volume of expandable member 111 via the outer catheter lumen, e.g., via a port on proximal portion 108B. In some examples, the port may be proximal to or defined by handle 104. In other examples, an inflation tube separate from the outer catheter body (e.g., extending through the outer catheter lumen) can be used to deliver an inflation fluid to the inner volume of expandable member 111 to expand expandable member 111 to an expanded state.

Introduction of the inflation fluid into the inner volume of expandable member 111 expands expandable member 111 from a relatively low profile collapsed configuration to an expanded configuration. The inflation fluid (also referred to as the “fluid”), is biocompatible. In some examples, the fluid may be a liquid such as sterile water or saline. In addition, in some examples, the fluid includes a contrast media, e.g., mixed with sterile water. The fluid can be removed from the inner volume of expandable member 111 to transition expandable member 111 from the expanded configuration into the collapsed configuration. The clinician may transform expandable member 111 between the expanded configuration and the collapsed configuration, e.g., to navigate catheter 102 between multiple target regions and to deliver therapeutic agents at the multiple target regions.

In some examples, catheter 102 is configured to accommodate a variety of vessel diameters. For example, renal vessels may have a diameter between about 3 mm and about 8 mm. Other vessels may have other diameters. Distal portion 108A may accommodate different vessel diameters because expandable member 111 is configured to expand to different diameters. In this way, a single catheter 102 may be used to deliver therapy to vessels with different diameters, e.g., diameters in a range of between about 2 mm and about 10 mm, such as in a range of about 3 mm to 8 mm.

Distal portion 108A of elongated body 108 is configured to be advanced within a hollow anatomical structure (e.g., a blood vessel) of a human patient to locate therapy delivery element 110 at a target region (e.g., a target treatment site) within or otherwise proximate to the hollow anatomical structure. For example, elongated body 108 may be configured to position therapy delivery element 110 within a blood vessel, a ureter, a urethra, a duct, an airway, or another naturally occurring lumen within the human body. The examples described herein focus on the anatomical structure being a blood vessel, such as a renal vessel, but it will be understood that similar techniques may be used with other hollow anatomical structures.

In certain examples, intravascular delivery of catheter 102 includes percutaneously inserting a guidewire (not shown in FIG. 1) into a vessel of a patient and moving elongated body 108 along the guidewire or another guide member until therapy delivery element 110 reaches a target treatment site (e.g., a renal artery). For example, catheter 102 can include a guidewire tube or another structure that defines a guidewire lumen configured to receive a guidewire for delivery of catheter 102 using over-the-wire (OTW) or rapid exchange (RX) techniques. In still other examples, catheter 102 can be configured for delivery via an inner catheter or sheath (not shown in FIG. 1), or other inner guide member (e.g., a guide catheter). In addition to or instead of an inner guide member, in some examples, catheter 102 is a steerable (e.g., with one or more pull wires connected to distal portion 108A and manipulatable by a clinician) or non-steerable catheter configured for use without a guidewire. Thus, in some examples, the inner member may be a navigation wire (e.g., a guidewire or the like), which can be disposed on an outer surface of elongated body 108 of catheter 102 or extend within an inner lumen of catheter 102.

Once at the target treatment site, the clinician may transition expandable member 111 from the collapsed configuration into the expanded configuration by at least introducing the fluid into the inner volume of expandable member 111, e.g., via the outer catheter lumen or via an inner tube disposed within the outer catheter lumen. Once expandable member 111 is in the expanded configuration, the clinician may operate a medical device to deliver therapeutic agent to the target treatment site via the one or more injection tubes disposed on expandable member 111. The therapeutic agent can, for example, provide or facilitate neuromodulation therapy at the target treatment site. For ease of description, the following discussion will be primarily focused on delivering therapeutic agents. It will be understood, however, that catheter 102 may include elements configured to deliver other types of therapies (e.g., needled chemical ablation, radiofrequency (RF) ablation, microwave ablation, ultrasound ablation, cryoablation, and the like).

FIG. 2A is a conceptual diagram illustrating a partial cross-sectional view of a proximal portion of catheter 102 of FIG. 1, the cross-section being taken along line A-A in FIG. 1. As illustrated in FIG. 2A, handle 104 may include connector 202 and housing 204 including delivery port 105. Housing 204 is directly or indirectly mechanically connected to proximal portion 108B of elongated body 108. Thus, a clinician can manipulate elongated body 108 by at least manipulating housing 204 (e.g., distally pushing housing 204 relative to a patient, proximally withdrawing housing 204 relative to the patient, rotating housing 204 about a longitudinal axis of elongated body 108, and the like). Elongated body 108 defines a catheter lumen in which one or more injection tubes 224 are positioned. Inner tube 214 is also positioned in the catheter lumen defined by elongated body 108.

Catheter 102 is configured to preferentially curve (also referred to as bend or flex in some examples) in one direction over at least one other direction, which can be a direction orthogonal to the direction of preferential curving or another direction. Catheter 102 may preferentially bend to facilitate orientation of the therapy delivery elements (e.g., one or more injection ports of injection tube(s) 224) of catheter 102 within vasculature of the patient. For example, based on knowledge of anatomy of the patient near a target treatment site, such as a location of one or more curves in the vasculature, elongated body 108 can be configured to preferentially curve in a particular direction and may self-orient injection ports away from certain anatomical features and/or towards certain anatomical features. Elongated body 108 can have an suitable configuration that facilitates the preferential curving of catheter 102. In some examples, elongated body 108 includes a structure, referred to herein as a self-orienting member 216, configured to preferentially curve and/or self-orient elongated body 108. For example, in some examples, as illustrated in FIG. 2A, elongated body 108 includes self-orienting member 216, which can be separate from and attached to elongated body 108 or may be integrally formed with elongated body 108.

Connector 202 may be attached to a proximal end 208 of housing 204 (e.g., a hub) and may be in fluid communication with injection tube(s) 224. Injection tube(s) 224 are configured to deliver a therapeutic agent to tissue of the patient via one or more injection ports defined by injection tube(s). For example, each injection tube 224 can define one or more injection ports, and, in some examples, the injection of some or all injection tubes 224 can be located at a distal portion of the respective injection tubes 224.

Connector 202 is configured to fluidically connect injection tube(s) 224 to a pressure source (e.g., a compressor, a pump, a gas canister or the like) and a therapeutic agent source of system 100 to facilitate delivery of the therapeutic agent to tissue of a patient via the one or more injection ports of injection tube(s) 224. Connector 202 may be permanently and/or removably fluidically connected to a proximal portion of injection tube(s) 224. In some examples, connector 202 is attached to housing 204 or can be separate from housing 204.

Housing 204 includes delivery port 105, which is in fluid communication with an inner volume of expandable member 111. A clinician can introduce and/or extract inflation fluid from within inner volume 232 (FIG. 2B) of expandable member 111 via delivery port 105. For example, delivery port 105 can be in fluid communication with the catheter lumen (either directly or indirectly), which defines a fluid pathway between delivery port 105 and inner volume 232 of expandable member 111. As another example, catheter 102 may further include an inner tube 214 that is in fluid communication with the inner volume of expandable member 111. Inner tube 214 may enter housing 204 and the catheter lumen through port 105 and the clinician may access inner tube 214 through injection port 105.

Handle 104 may additionally include one or more components not pictured in FIG. 2A including, but are not limited to, structures to facilitate delivery of a therapeutic agent to tissue via therapy delivery element 110. For example, handle 104 can include a trigger to initiate delivery of a therapeutic agent into injection tube(s) 214, a visualization window, a safety collar, a cam clamp, or the like. The clinician may operate the one or more components to deliver the therapeutic agent to, to inspect the therapeutic agent within handle 104, prevent unintended delivery of the therapeutic agent, or the like. In some examples, elongated body 108 may be secured to housing 204, e.g., via a cam clamp disposed on housing 204.

FIG. 2B is a conceptual diagram illustrating a cross-sectional view of a distal portion of catheter 102 of FIG. 1, the cross-section being taken along line A-A in FIG. 1. Distal portion 108A of elongated body 108 may be connected to a proximal end of expandable member 111. In some examples, as illustrated in FIG. 2B, elongated body 108 includes self-orienting member 216. Guidewire tube 212, inner tube 214, and at least a portion of injection tube(s) 124 may be disposed within a distal portion of catheter lumen 211.

Guidewire tube 212 may extend through expandable member 111 and may be connected to distal end 112 of catheter 102. Guidewire tube 212 defines a guidewire lumen configured to receive one or more inner members (e.g., a guidewire, a guiding member, or the like). A clinician may navigate catheter 102 through vasculature of the patient with the aid of the inner member received in guidewire tube 212. In some examples, a proximal opening to guidewire tube 212 is at handle 104 and/or a point proximal to handle 104. In other examples, a proximal opening to guidewire tube 212 is at a point distal to handle 104 (e.g., via a RX port). Guidewire tube 212 may include a relatively flexible material (e.g., a relatively soft polymer such as high-density polyethylene (HDPE)) and can include a lubricious inner lumen. In some examples, guidewire tube 212 may includes a plurality of layers (e.g., two layers, three, or more than three layers). In some examples, an inner layer (e.g., an innermost layer) of guidewire tube 212 includes the relatively flexible material (e.g., HDPE) and an outer layer (e.g., an outermost layer) of guidewire tube 212 includes a nylon including, but is not limited to, PEBAX (e.g., PEBAX 55 or PEBAX 63). The nylon may include, for example, a softer PEBAX (e.g., PEBAX 40 or PEBAX 45) and/or a harder PEBAX (e.g., PEBAX 70 or PEBAX 72).

Injection tube(s) 224 is disposed within catheter lumen 211. Injection tube(s) 224 may exit catheter lumen 211 at a point proximal to expandable member 111 and an external portion 226 of injection tube(s) 224 may be disposed on an outer surface of expandable member 111. External portion 226 of each injection tube defines one or more injection ports 228. In some examples, when expandable member 111 is in the expanded configuration and in a blood vessel of a patient, expandable member 111 positions external portion 226 and/or injection ports 228 in apposition with a wall of the blood vessel. When expandable member 111 transitions to the collapsed configuration, external portion 226 may remain connected to the outer surface of expandable member 111 and may contract radially inwards away from the blood vessel wall alongside expandable member 111. In some examples, external portion 226 may include one or more radiopaque markers to facilitate visualization of injection port(s) 228 and aid in delivery of the therapy to tissue of the patient.

As illustrated in FIG. 2B, at least a portion of elongated body 108 includes self-orienting member 216. A distal end 222 of self-orienting member 216 may be disposed within another portion of elongated body 108 (e.g., outer catheter body 218 of elongated body 108). Self-orienting member 216 may be advanced and/or retracted within an inner lumen of outer catheter body 218. Outer catheter body 218 may be connected to a proximal end of expandable member 111. In other examples, self-orienting member 216 may be permanently connected to elongated body 108.

Self-orienting member 216 is configured to place external portion 226 and injection port 228 of injection tube(s) 224 at a predictable orientation when placed within the vasculature of the patient by at least preferentially bending while elongated body 108 is disposed within the vasculature of the patient. For example, when a portion of elongated body 108 including self-orienting member 216 is positioned around a curve in a blood vessel, self-orienting member 216 causes elongated body 108 to curve in a predictable direction to enable injection port 228 to orient away from certain anatomical features and/or towards certain anatomical features. For example, self-orienting member 216 may position external portion 226 and injection port 228 away from a particular anatomical feature of the patient when distal portion 108A of catheter 102 is disposed within a blood vessel of the patient. In some examples, self-orienting member 216 preferentially curves at least distal portion 108A of catheter 102 when distal portion 108A is disposed within the vasculature. In some examples, the orientation of external portion 226 and injection port 228 may include, e.g., a rotational orientation of external portion 226 and injection port 228 within the blood vessel. The rotation orientation may be defined by a radial position of external portion 226 and injection port 228 along the cross-section of the blood vessel wall.

Self-orienting member 216 has any suitable configuration that facilitates preferential bending of elongated body 108 when elongated body 108 extends around curvature in vasculature of a patient. In some examples, a distal portion 222 of self-orienting member 216 defines a taper in a distal direction. Distal portion 222 of self-orienting member 216 is configured to preferentially curve by at least more easily curving in a first direction and resisting curvature in a second direction, as described in greater detail below. In some examples, as illustrated in FIG. 2B, self-orienting member 216 includes a hypotube and distal portion 222 of is a spline attached to a distal end of the hypotube. For example, the spline may be welded to the distal end of the hypotube or may be integrally formed from the hypotube, such as by laser cutting the hypotube to form the spline.

In some examples, self-orienting member 216 is formed from a shape-memory material such as, but is not limited to, a shape-memory metal alloy such as Nitinol. In other examples, self-orienting member 216 is formed from a non-shape-memory material and the configuration (e.g., geometry) of self-orienting member 216 configures it to preferentially curve in one direction over at least one other direction.

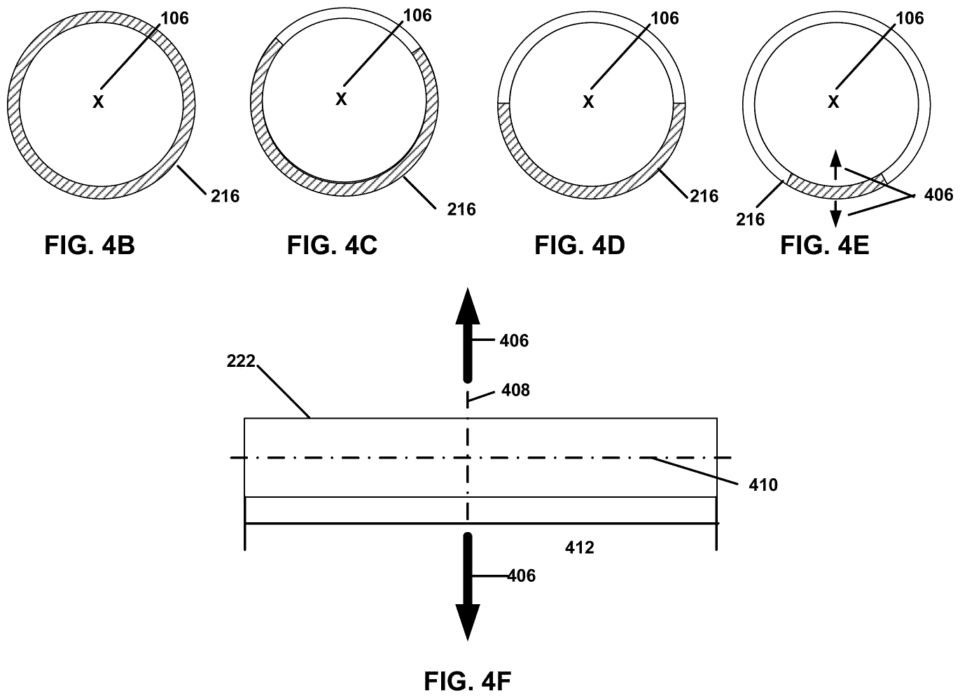

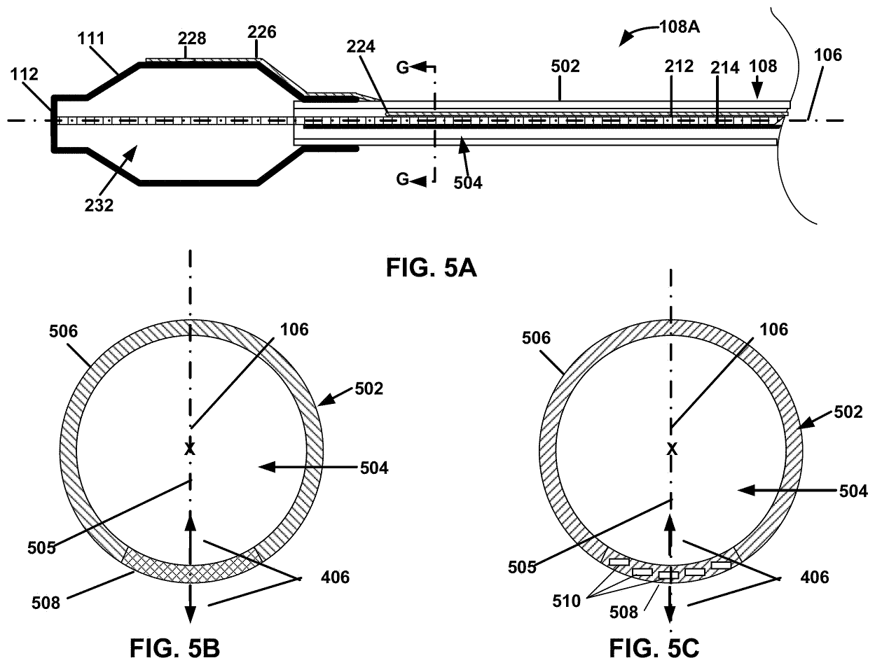

In examples in which catheter 102 does not include self-orienting member 216 but is still configured to preferentially curve in one direction over another, a cross-sectional composition of elongated body 108 and/or outer catheter body 218 enables elongated body 108 to self-orient and/or preferentially curve, e.g., as discussed in further detail below with reference to FIGS. 4A-5C.

In some examples, the preferential curvature and/or self-orientation of elongated body 108 is user-selected. For example, a clinician can select an elongated body 108 from a plurality of available elongated bodies having different predetermined orientations of curvature relative to injection ports 228 of the catheter based at least in part on the preferential curvature of the particular elongated body. The clinician may determine the preferential curvature and/or the self-orientation based at least in part on a location of the target treatment site in the patient, the insertion site for catheter, and/or anatomical features to be avoided. For example, the clinician may select a catheter that preferentially curves, self-orients, or has a predetermined orientation that orients injection ports 228 away from the renal vein for a neuromodulation therapy directed at wall tissue of a renal vessel.

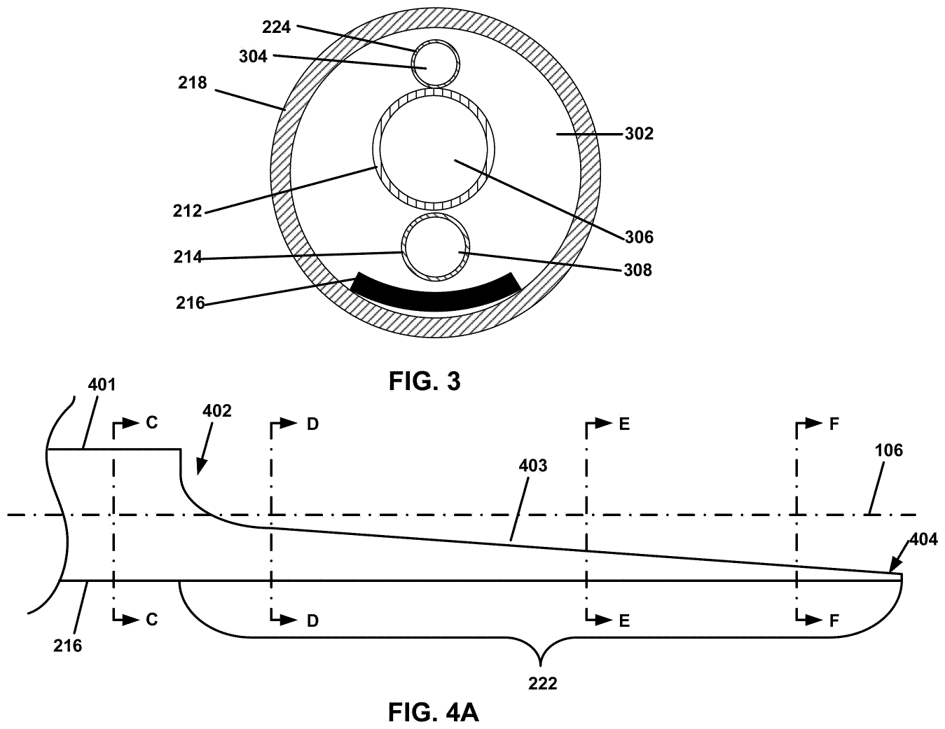

FIG. 3 is a conceptual diagram illustrating a cross-sectional view of distal portion 108A of catheter 102 of FIG. 2B, the cross-section being taken along line B-B in FIG. 2B in a direction orthogonal to longitudinal axis 106. Outer catheter body 218 defines outer catheter lumen 302. Injection tube(s) 224, guidewire tube 212, inner tube 214, and self-orienting member 216 are disposed within outer catheter lumen 302 in the example shown in FIG. 3. Outer catheter body 218 may have any suitable thickness, defined in a radial direction between the outer surface of outer catheter body 218 and the inner surface of outer catheter body 218 defining outer catheter lumen 302. In some examples, outer catheter body 218 has a thickness of about 0.05 millimeters (mm) to about 0.3 mm (e.g., about 0.002 inches to about 0.010 inches).

Injection tube(s) 224 define injection tube lumen 304. Injection tube lumen 304 may retain a therapeutic agent and is in fluid communication with injection port 228. In some examples, injection tube lumen 304 of a single injection tube 224 is in fluid communication with multiple external portions 226 disposed on expandable member 111. That is, an injection tube 224 may split into multiple external portions 226 in some examples. In these examples, the external portions 226 may be considered to be respective injection tubes 224 each defining one or more injection lumens. In other examples, each injection tube 224 defines a respective injection lumen 304 that remains fluidically separate from an injection lumen 304 of another injection tube 224 throughout the entire length of the injection tube 224. Injection tubes 224 can have any suitable configuration that facilitates relatively high pressure fluid injection, as well as facilitates delivery of catheter 102 through vasculature of a patient to a target site. In some examples, injection tube(s) 224 may withstand at least 18 MPa and may be resistant to frequent changes in pressure.

Guidewire tube 212 defines guidewire lumen 306. As discussed above, a clinician may access guidewire lumen 306, e.g., via a rapid exchange (RX) port disposed along elongated body 108 or at point closer to or at a proximal end of catheter 102. Inner tube 214 defines inner tube lumen 308, which, as discussed above, can be used to deliver an inflation fluid into and remove inflation fluid from inner volume 232 of expandable member 111. In some examples, however, catheter 102 does not include inner tube 214 and the inflation fluid is delivered to inner volume 232 of expandable member via outer catheter lumen 302.

FIG. 4A is a conceptual diagram illustrating an example self-orienting member 216 of catheter 102 of FIGS. 2A and 2B. Self-orienting member 216 includes distal portion 222 tapering from first point 402 to distal end 404, such that distal portion 222 lowers in profile in a distal direction. In some examples, self-orienting member 216 includes a first member 401 (e.g., a hypotube) extending to first point 402 and a second member 403 (e.g., a spline) attached to first member 401 at first point 402 and extending to distal end 404. In other examples, however, first member 401 is cut to define second member (e.g., a cutout in the hypotube) may define first point 402.

Self-orienting member 216 can have any suitable dimension, which may depend on the intended target tissue site for catheter 102, the type of therapy being delivered, or the like. For example, in some examples, such as some examples in which catheter 102 is configured to deliver a therapeutic agent to tissue via an intravascular location, self-orienting member 216 may have an outer diameter of about 0.5 mm to about 1.5 mm (e.g., about 0.02 inches to about 0.06 inches) and an inner diameter of 0.38 mm to about 1.4 mm (e.g., about 0.015 inches to about 0.055 inches). Self-orienting member 216 can have, for example, a wall thickness (e.g. a thickness of self-orienting member 216 between the outer surface of self-orienting member 216 and an inner surface of self-orienting member 216 defining the inner lumen) of about 0.0125 mm to about 0.077 mm (e.g., about 0.0005 inches to about 0.003 inches). In some examples, a relatively larger diameter self-orienting member 216 can include one or more features to increase the flexibility of the self-orienting tube 216. For example, self-orienting member 216 can include one or more cuts (e.g., a spiral cut, a plurality of radial cuts, or the like) to increase its flexibility along its length.

In some examples, self-orienting member 216 has an overall length of about 10 centimeters (cm) to about 35 cm (e.g., about 3.9 in to about 13.8 in). In some examples, self-orienting member 216 may be contained in a length of elongated body 108 of about 10 cm to about 35 cm (e.g., about 3.9 in to about 13.8 in) proximal to the proximal end of expandable member 111, e.g., to facilitate positioning of injection ports 228 in apposition with the vessel wall in the predictable orientation. In some examples, self-orienting member 216 can be shorter, e.g., have an overall length of about 4 cm to about 5 cm (e.g., about 1.57 in to about 1.97 in). The overall length of self-orienting member 216 can be measured from its proximal-most end to its distal-most end.

FIG. 4B is a conceptual diagram illustrating a cross-sectional view of example self-orienting member 216 of FIG. 4A, the cross-section being taken along line C-C in FIG. 4A. FIG. 4C is a conceptual diagram illustrating a cross-sectional view of example self-orienting member 216 of FIG. 4A, the cross-section being taken along line D-D in FIG. 4A. FIG. 4D is a conceptual diagram illustrating a cross-sectional view of example self-orienting member 216 of FIG. 4A, the cross-section being taken along line E-E in FIG. 4A. FIG. 4E is a conceptual diagram illustrating a cross-sectional view of example self-orienting member 216 of FIG. 4A, the cross-section being taken along line F-F in FIG. 4A. FIG. 4F is a conceptual diagram illustrating another cross-sectional view of example self-orienting member 216 of FIG. 4A, taken along line F-F in FIG. 4A. Each of the cross-section shown in FIGS. 4B-4F is taken in a direction orthogonal to longitudinal axis 106.

As FIGS. 4B-4F illustrate, as self-orienting member 216 tapers from first portion 402 towards distal end 404, the cross-section of self-orienting member 216 transforms. The cross-section of self-orienting member 216 transitions from an annular cross-section, as shown in FIG. 4B, to a partially annular cross-section, as shown in FIGS. 4C-E, to a partially annular cross-section substantially resembling a rectangular cross-section, as illustrated in FIG. 4F. In other examples, the partially annular cross-section may have a curved cross-section at distal end 404. The partially annular cross-section of self-orienting member 216 in FIG. 4D subtends a smaller angle than the partially annular cross-section of self-orienting member 216 in FIG. 4C, and the partially annular cross-section of self-orienting member 216 in FIG. 4E subtends a smaller angle than the cross-section shown in in FIG. 4D.

The cross-section may have a largest dimension, referred to herein as a width 412 (e.g., along major axis 410), of about 0.30 mm to about 0.80 mm (e.g., about 0.010 inches to about 0.0030 inches). In some examples, the cross-section may have width 412 of between about 0.25 mm to about 0.8 mm. In some examples, once self-orienting member 216 has tapered to the cross-section shown in FIG. 4F, self-orienting member 216 may preferentially curve and/or deflect in response to forces 406 but may maintain rigidity in response to forces orthogonal to forces 406. Self-orienting member 216 is configured to maintain rigidity in response to forces orthogonal to forces 406, which can help maintain a desirable level of pushability of self-orienting member 216 during navigation of elongated body 108 to a target tissue site. Self-orienting member 216 may be configured to maintain rigidity in response to forces orthogonal to forces 406, which can help ensure that self-orienting member 216 preferentially curves and/or self-orients into the predictable orientation.

As shown in FIG. 4F, distal end 222 of self-orienting member 216 defines major axis 410 and minor axis 408. Major axis 410 may be parallel to a longer edge of the cross-section of distal end 222 and minor axis 408 may be orthogonal to major axis 410. As illustrated in FIG. 4F, self-orienting member 216 may preferentially curve, deflect, and/or deform in a direction of minor axis 408 and resist bending in a direction of major axis 410, such as by maintaining rigidity in a direction of major axis 410.

In some examples, force 406 is applied, e.g., by interaction between elongated body 108 and vessel wall, at a centerline of a cross-section of self-orienting member 216. In some examples, force 406 is applied at any position along self-orienting member 216 and in the direction of minor axis 408.

As discussed above, catheter 102 can be configured to preferentially flex in one direction over another by including self-orienting member 216 disposed within a lumen (e.g., outer catheter lumen 302) defined by elongated body 108. In other examples, as discussed in greater detail in FIGS. 5A-5C, catheter 102 can be configured to preferentially flex in one direction over at least one other direction by including one or more self-orienting structures within elongated body 108 to configure elongated body 108 to preferentially flex in one direction over at least one other direction or otherwise configuring elongated body 108.

FIG. 5A is a conceptual diagram illustrating a cross-sectional view of another example distal portion 108A of catheter 102 of FIG. 1, the cross-section being taken along line A-A in FIG. 1. Elongated body 108 includes outer catheter body 502 defining outer catheter lumen 504. Outer catheter body 502 may be connected to a proximal end of expandable member 111. Outer catheter lumen 504 may contain injection tube(s) 224, guidewire tube 212, and/or inner tube 214. Outer catheter body 502 has a structure that preferentially curves elongated body 108 in one direction over another as catheter 102 traverses a curvature in a blood vessel of a patient. In some examples, outer catheter body 502 includes structures that causes outer catheter body 502 to self-orient within a blood vessel, e.g., into a predetermined orientation. The structures of outer catheter body 502 may cause outer catheter body 502 to self-orient as catheter 102 traverses a curvature in the blood vessel of the patient. As illustrated in FIG. 5A, outer catheter body 502 of elongated body 108 may be a self-orienting member.

FIG. 5B is a conceptual diagram illustrating a cross-sectional view of an example of distal portion 108A of catheter 102 of FIG. 5A, the cross-section being taken along line G-G in FIG. 5A. FIG. 5C is a conceptual diagram illustrating a cross-sectional view of another example of distal portion 108A of catheter 102 of FIG. 5A, the cross-section being taken along line G-G in FIG. 5A. The cross-section view illustrated in FIGS. 5B and 5C are orthogonal to longitudinal axis 106 of catheter 102. While FIGS. 5B and 5C only illustrate a cross-section of outer catheter body 502, injection tube(s) 224, guidewire tube 212, and/or inner tube 214 may be disposed within outer catheter body 502 (e.g., within outer catheter lumen 504) in accordance with one or more other examples described herein.

As illustrated in FIG. 5B, outer catheter body 502 defines outer catheter lumen 504 and includes first circumferential portion 506 and second circumferential portion 508. Together, first and second circumferential portions 506, 508 subtend fully around the perimeter of the cross-section outer catheter body 502. In other examples, catheter body 502 can include one or more additional circumferential portions that together with first and second circumferential portions 506, 508 subtend fully around the perimeter of the cross-section outer catheter body 502. Outer catheter body 502 is configured to, in response to force 406, preferentially curve or deflect along axis 505. Outer catheter body 502 may resist curvature and/or maintain rigidity in a direction orthogonal to longitudinal axis 106. Outer catheter body 502 may have a width from an outer surface to outer catheter lumen of about 0.05 mm to about 0.15 mm (e.g., about 0.002 inches to about 0.005 inches).

In the example shown in FIG. 5B, first circumferential portion 506 extends along a majority of an outer perimeter of the annular cross-section of outer catheter body 502. First portion 506 and second portion 508 are configured to have different properties, e.g., different hardnesses or flexibilities, to facilitate the preferential flexing of outer catheter body 502 in one direction over another. In some examples, first portion 506 and second portion 508 both include biocompatible polymers including, but are not limited to, Pebax (e.g., Pebax 55 or the like), but second portion 508 includes a biocompatible polymer that is harder than the material in both first portion 506 such as, but is not limited to, a stiffer Pebax (e.g., Pebax 63 or the like). In some examples, first portion 506 and second portion 508 may include a braid formed from polymer, where the biocompatible polymers form individual strands that are braided together to form outer catheter body 502. In second portion 508, the strands of the relatively softer polymer may be braided alongside strands of the relatively stiffer polymer to form second portion 508.

In other examples, first and second circumferential portions 506, 508 can exhibit different properties to enable catheter 102 to preferentially curve in one direction over another using other techniques. For example, as illustrated in FIG. 5C, in some examples, second portion 508 includes a plurality of inserts 510 configured to increase the stiffness of second portion 508 relative to first portion 506. Inserts 510 can be, for example, longitudinal strands of a relatively stiff (compared to first circumferential portion 506) material. The longitudinal strands can be oriented substantially linearly or nonlinearly in some examples. In at least this example, first portion 506 and second portion 508 may both include one or more relatively soft materials including, but is not limited to, a relatively soft biocompatible polymer (e.g., Pebax 55 or the like). Second portion 508 may also include inserts 510 arranged parallel to longitudinal axis 106. Inserts 510 may include a stiffer material sch as, but is not limited to, a metal alloy (e.g., Nitinol or the like).

In response to force 406, second portion 508 is configured to curve and/or deflect towards and/or away from longitudinal axis 106. Second portion 508 may also resist curvature and/or deflection in a direction orthogonal to longitudinal axis 106.

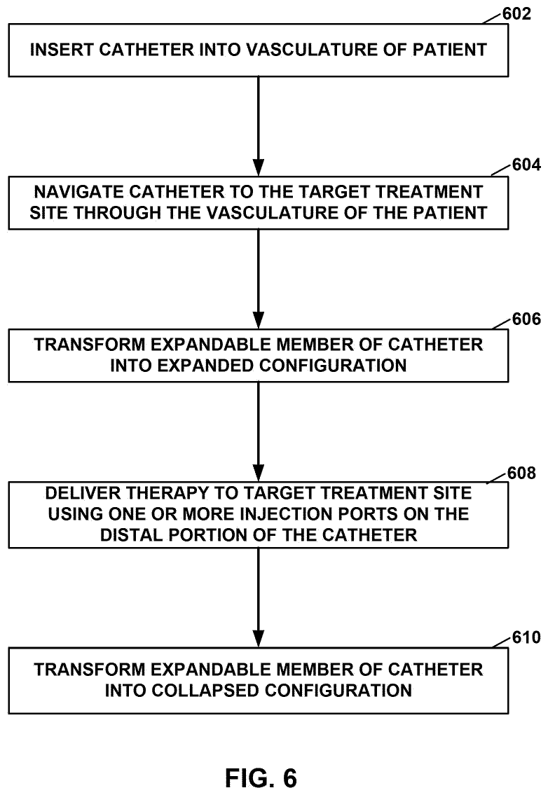

FIG. 6 is a flow diagram illustrating an example process of delivering therapy to a patient using a catheter (e.g., catheter 102) configured to preferentially curve. While FIG. 6 is primarily described with respect to therapy delivery to a target treatment site within a renal vessel of a patient, the example processes may be used for other target treatment sites and/or for other cavities within the patient. In some examples, catheter 102 may be disposed in the renal artery and may preferentially curve and/or self-orient away from a renal vein and prevent unintended therapy delivery to the renal vein. This may help reduce the likelihood that a therapeutic agent or other fluid will be delivered to unintended tissue sites (e.g., the renal vein) in the patient. Catheter 102 may also reduce an amount of time needed for the clinician to align and orientate catheter 102 within the vasculature and reduce an overall medical procedure time.

A clinician may insert catheter 102 into vasculature of a patient (602). The clinician may make an incision in the skin of the patient at an insertion site on the patient to reach a blood vessel of the patient. The clinician may then insert at least distal portion 108A of catheter 102 into the blood vessel. In some examples, the clinician may insert catheter 102 into the vasculature without positioning expandable member 111 and/or injection tube(s) 224 in a user-determined orientation before insertion into the body of the patient. Once inserted into the vasculature, elongated body 108 of catheter 102 may automatically self-orient and/or preferentially curve expandable member 111 and/or injection tube(s) 224 into a desired configuration that enables more targeted delivery of a therapeutic agent to a target tissue site. Elongated body 108 may automatically self-orient and/or preferentially curve due to self-orienting member 216 within elongated body 108 and/or the structure of elongated body 108.

The clinician may navigate catheter 102 to the target treatment site through the vasculature of the patient (604). The clinician may navigate catheter 102 using one or more imaging techniques such as X-ray imaging, fluoroscopy, or the like. In some examples, clinician advances catheter 102 over a guide member disposed within the vasculature (e.g., via OTW technique, rapid exchange technique, or the like). When the clinician encounters curvatures within the vasculature of the patient, the clinician may advance distal portion 108A around the curvature and elongated body 108 will preferentially curve and/or self-orient expandable member 111 and/or injection tube(s) 224 into the user-determined orientation. The clinician may advance catheter 102 within the vasculature until distal portion 108A reaches the target treatment site. In some examples, with regard to renal neuromodulation, the clinician may advance catheter 102 from a trans-radial entry point on the patient to a renal artery of the patient.

The clinician may align expandable member 111 and/or injection ports 228 of injection tube(s) 224 with target treatment site, e.g., by advancing or retracting catheter 102 within the vasculature. Expandable member 111 and/or injection ports 228 may be automatically oriented in a preferred direction relative to a target tissue site due to elongated body 108. For example, the preferred direction may be such that injection ports 228 are oriented away from one or more anatomical features near the target treatment site. For example, if the target treatment site is located within the renal artery of the patient, the user-determined orientation may face injection ports 228 away from the renal vein.

The clinician may transform expandable member 111 of catheter 102 into an expanded configuration (606). The clinician may insert an inflation fluid into inner volume 232 of expandable member 111. In some examples, the clinician inserts the inflation fluid into inner volume 232 via inner tube 214 (e.g., via a proximal end of inner tube 214 disposed within or near handle 104) and/or a catheter lumen of elongated body 108 (e.g., catheter lumen 302, outer catheter lumen 504, or the like). The clinician may insert inflation fluid into expandable member 111 until expandable member 111 is in the expanded configuration. As expandable member 111 transitions to the expanded configuration, an outer surface of expandable member 111 may expand radially outwards of elongated body 108. At the expanded configuration, expandable member 111 may place injection ports 228 in apposition with wall tissue at the target treatment site.

The clinician may deliver therapy to a target treatment site using one or more injection ports 228 on distal portion 108A of catheter 102 (608). The clinician may actuate catheter delivery system 100 to transmit a therapeutic agent at relatively high pressure (e.g., between about 5 MPa to about 18 MPa) through injection tube(s) 224. The pressurized therapeutic agent may exit injection ports 228 and penetrate the wall tissue in apposition with the injection ports 228. In some examples, with regard to renal neuromodulation, the clinician may deliver the therapeutic agent to a portion of the wall tissue of the renal artery that faces away from the renal vein. The clinician may continue to deliver the therapeutic agent to the target treatment site until the clinician determines that a user-determined amount of the therapeutic agent has been administered to the patient.

The clinician may transform expandable member 111 of catheter 102 into a collapsed configuration (610). The clinician may remove inflation fluid from inner volume 232 of expandable member 111. The clinician may remove the inflation fluid via inner tube 214 and/or the catheter lumen (e.g., catheter lumen 302, outer catheter lumen 504, or the like). Once expandable member 111 is in the collapsed configuration, the clinician may remove catheter 102 from the vasculature and/or navigate distal portion 108A to a second target treatment site within the patient and deliver therapy to the second treatment site.

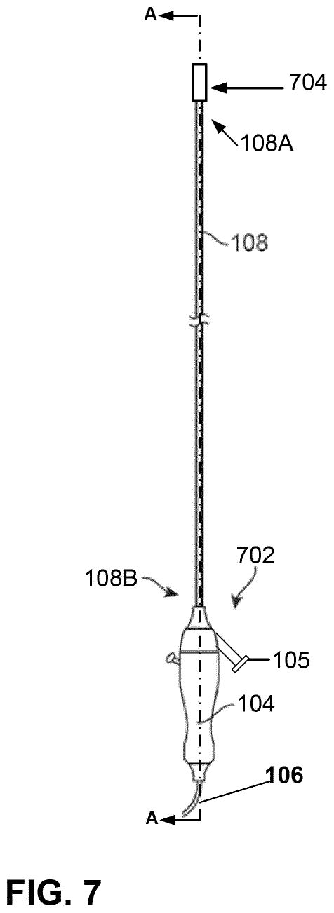

FIG. 7 is a partial schematic illustration of another example catheter 702 that includes a neuromodulation element 704. While some example catheters described in this disclosure (e.g., catheter 102 of FIG. 1) includes therapy delivery element 110 having an expandable member (e.g., expandable member 111), other example catheters, as illustrated by catheter 702 in FIG. 7, include therapy delivery element 704 without expandable member 111 and may be configured to preferentially curve and/or self-orient within vasculature of a patient. FIG. 7 also describes an example in which therapy delivery element 704 can be configured to deliver a therapeutic agent or other types of neuromodulation therapy, such as, but not limited to, RF energy, microwave energy, ultrasound energy, cryoablation therapy, or the like.

Therapy delivery element 704 is configured to deliver neuromodulation therapy to tissue of the patient and may include, but is not limited to, one or more energy delivery elements (e.g., electrodes, ultrasound transducers, or the like), a fluid delivery element (e.g., a needle), or a cryogenic element. Therapy delivery element 704 may be configured to deliver neuromodulation therapy to a target treatment site, e.g., that extends only part way or fully around the circumference of a blood vessel. For example, the area of influence of therapy delivered by therapy delivery element 704 may extend less than 360 degrees around the circumference of the blood vessel. The area of influence may correspond to a therapy delivery region on therapy delivery element 704. Therapy delivery region may correspond to, e.g., an active area of an energy delivery element or a cryogenic element, a placement of an injection device (e.g., a needle) for a fluid delivery element, or the like. In some examples, the therapy delivery region extends partially (e.g., less than 360 degrees) around a circumference of neuromodulation element 704.

Catheter 702 is configured to preferentially curve (also referred to as bend) in one direction over at least one other direction, e.g., in accordance with other similar structures in catheter 102 as described herein. Catheter 702 may preferentially bend to facilitate orientation of the areas of influence of therapy delivery element 704 within the vasculature of the patient. For example, based on knowledge of anatomy of the patient near a target treatment site, such as a location of one or more curves in the vasculature, elongated body 108 of catheter 702 can be configured to preferentially curve in a particular direction and may self-orient neuromodulation element 704 away from certain anatomical features and/or towards certain anatomical features, e.g., in accordance with other examples described herein.

For example, catheter 702 may preferentially bend and/or self-orient to orient a needle of therapy delivery element 704 away from certain anatomical structures of the patient (e.g., the renal vein) and towards the target treatment site within a blood vessel (e.g., within the renal artery). In some examples, catheter 702 may preferentially bend to orient an energy-active area of an ultrasound transducer, an electrode, or another energy delivery element away from certain anatomical structures of the patient and towards the target treatment site. In some examples, catheter 702 may preferentially bend in a particular direction to orient an active area of a cryogenic delivery element away from certain anatomical features of the patient and towards the target treatment site.

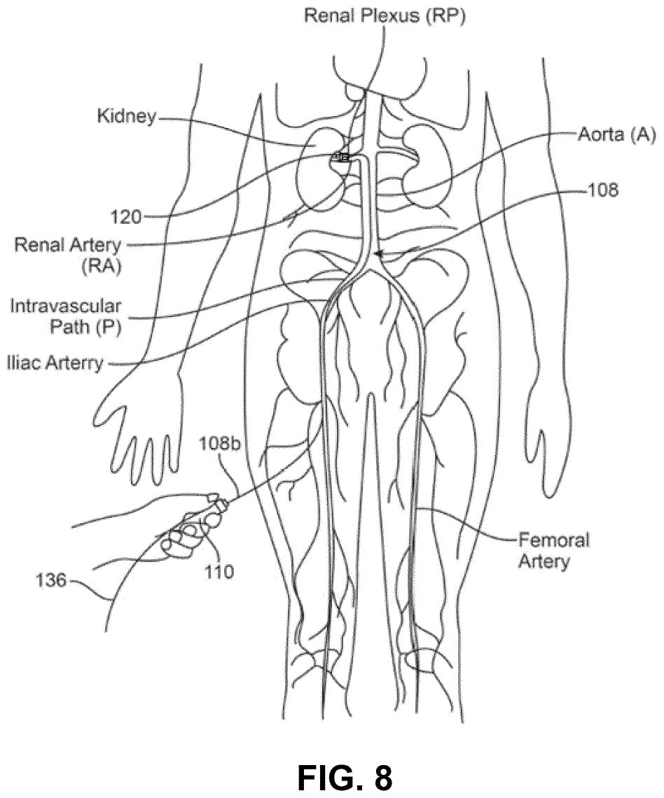

FIG. 8 is a conceptual illustration of an example process for accessing a renal artery and modulating renal nerves with the catheter of FIG. 1. While FIG. 8 illustrates the use of catheter 102 for renal neuromodulation, catheter 102 may be used for other therapies and treatments within another blood vessel or other hollow anatomical body within the human body. Catheter 102 is configured to delivery energy (e.g., radiofrequency energy, ultrasound energy, electrical stimulation energy, or the like) to one or more target treatment sites within a renal vessel. Catheter 102 provides access to the renal plexus (RP) through an intravascular path (P), such as a percutaneous access site in the femoral (illustrated), brachial, radial, or axillary artery to the target treatment sites within a respective renal artery (RA). By manipulating proximal portion 108B of elongated body 108 from outside the intravascular path (P), a clinician may advance at least distal portion 108A of elongated body 108 through the sometimes-tortuous intravascular path (P) and remotely manipulate distal portion 108A (FIG. 1) of elongated body 108. Distal portion 108A may be remotely manipulated by the clinician using the handle 104.

In the example illustrated in FIG. 8, distal portion 108A is delivered intravascularly to the treatment site using an inner member 136 in an over-the-wire (OTW) technique. Inner member 136 may be internal to catheter 102 (e.g., a guide wire, inner catheter, or the like) or external to catheter 102 (e.g., an outer sheath or the like). In some examples, inner member 136 is a navigation wire. Catheter 102 may define a passageway for receiving inner member 136 for delivery of catheter 102 using either an OTW or an RX technique. At the treatment site, inner member 136 can be at least partially withdrawn or removed relative to catheter 102 and distal portion 108A can transform into an expanded configuration (e.g., a helical configuration, a spiral configuration, or the like) for delivering ultrasound energy. In other examples, elongated body 108 may be self-steerable such that therapy delivery element 110 may be delivered to the target treatment site without the aid of inner member 136.

Renal nerve modulation is the partial or complete incapacitation or other effective disruption of nerves supplying the kidneys (e.g., nerves terminating in the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to add beneficial effects to hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced or induced in another suitable manner through the delivery of energy (RF energy, ultrasound energy, microwave energy, or the like). The target treatment site can be within or otherwise proximate to a renal lumen (e.g., a renal artery, a ureter, a renal pelvis, a major renal calyx, a minor renal calyx, or another suitable structure), and the target treatment site can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target treatment devices and associate methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operated through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up-and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

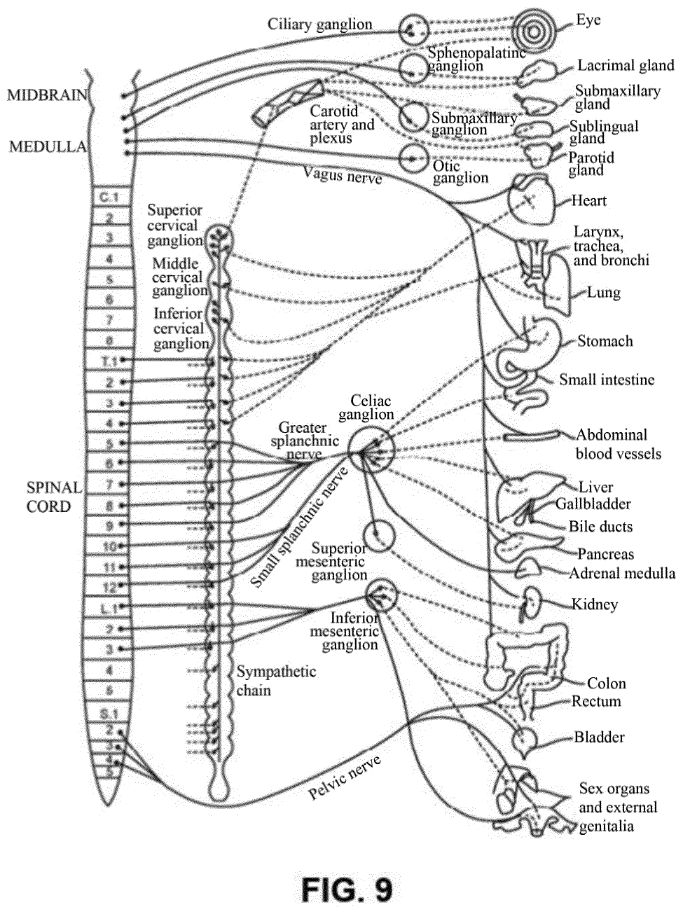

FIG. 9 is a conceptual illustration of an example sympathetic nervous system (SNS) illustrating how the brain communicates with the body via the SNS. As shown in FIG. 9, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

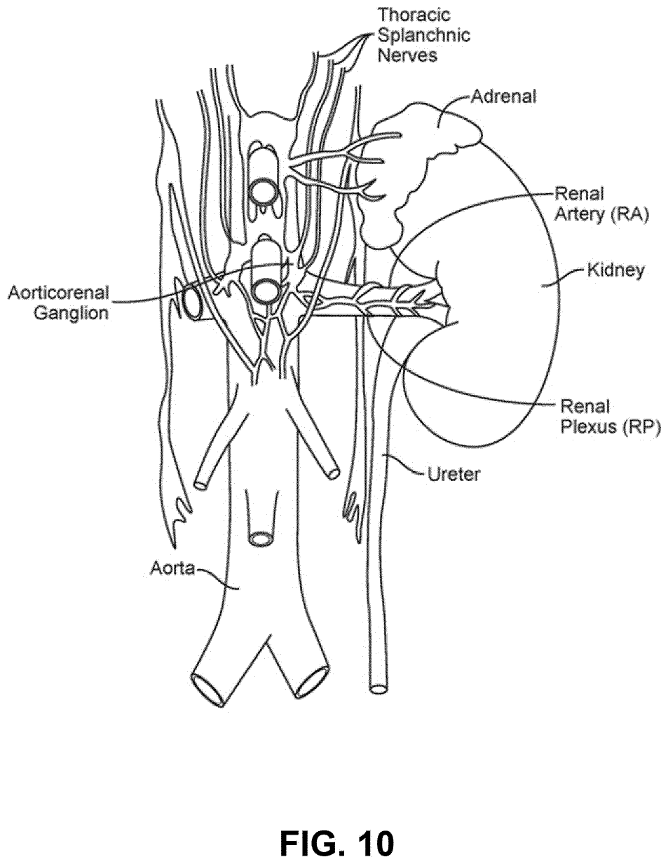

FIG. 10 is an enlarged anatomic view of nerves innervating a left kidney to form the renal plexus surrounding the left renal artery. As FIG. 10 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the renal blood vessels, the juxtaglomerular apparatus, and the renal tubules. Stimulation of the renal sympathetic nerves cause increased renin release, increased sodium (Na) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication.

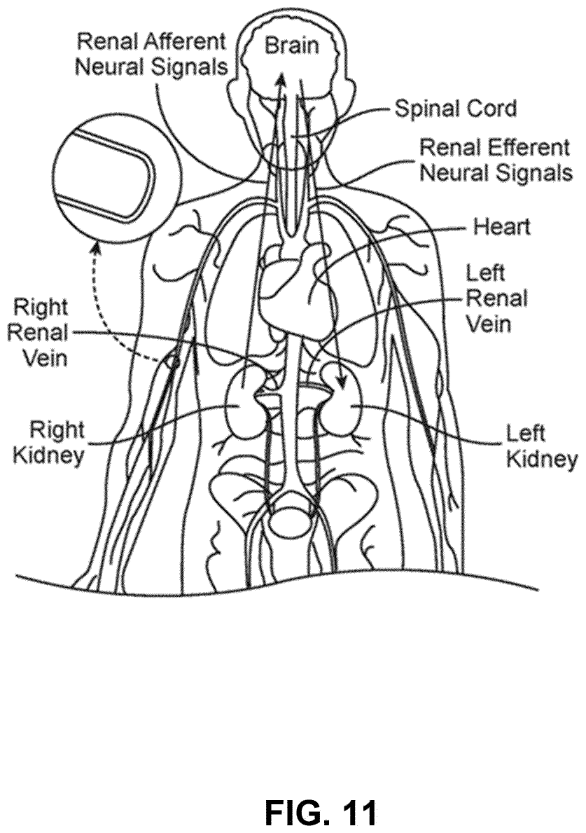

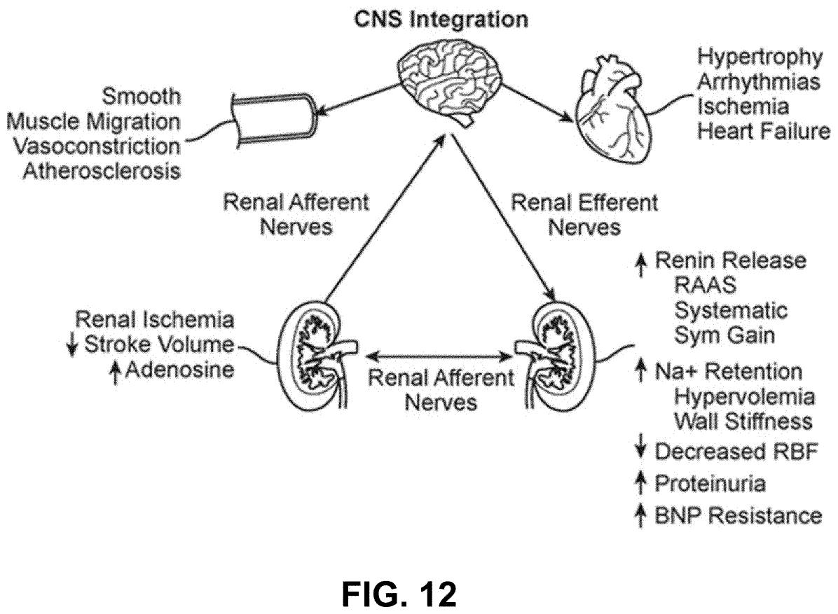

FIG. 11 is an anatomic view of a human body depicting neural efferent and afferent communication between the brain and kidneys. FIG. 12 is a conceptual view of a human body depicting neural efferent and afferent communication between the brain and kidneys. As shown in FIGS. 11 and 12, the afferent communication might be from kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 11. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

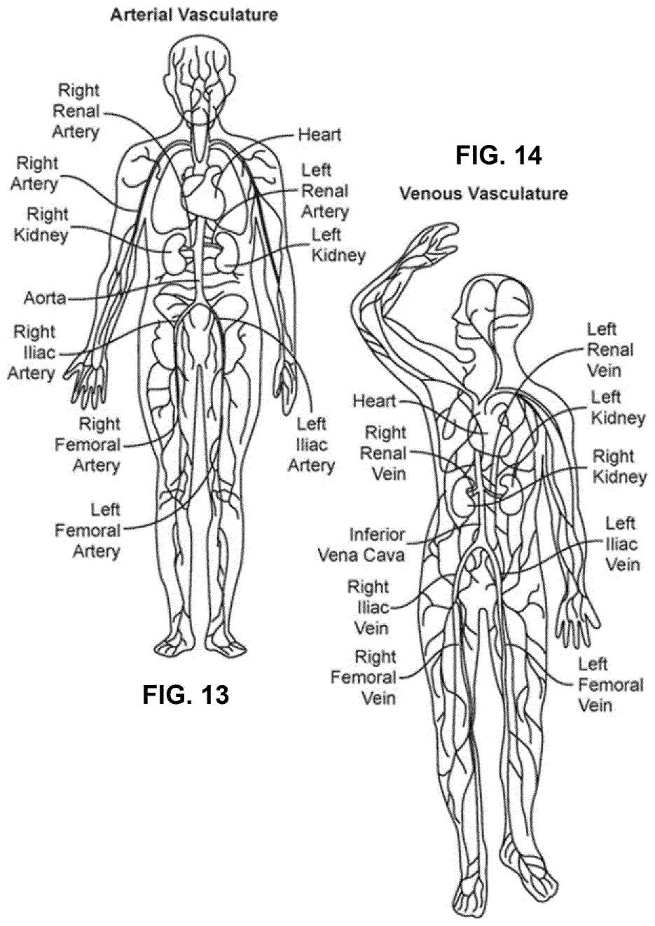

In accordance with the present technology neuromodulation of a left and/or right renal plexus (RP), which is intimately associated with a left and/or right renal artery, may be achieved through intravascular access. FIG. 13 is an anatomic view of the arterial vasculature of a human. As FIG. 13 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

FIG. 14 is an anatomic view of the venous vasculature of a human. As FIG. 14 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base on the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and therapy delivery elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Various aspects of the disclosure have been described, such as in the following examples. These and other aspects are within the scope of the claims.

Example 1: a catheter comprising: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to preferentially curve in a first direction over a second direction when the catheter is positioned in a curvature in vasculature of a patient.

Example 2: the catheter of example 1, wherein the catheter body comprises a self-orienting member configured to cause the preferential curvature.

Example 3: the catheter of example 2, wherein the self-orienting member is configured to deform relative to a first axis of the self-orienting member, and wherein the self-orienting member is configured to maintain rigidity relative to a second axis of the self-orienting member, the second axis being orthogonal to the first axis.

Example 4: the catheter of example 3, wherein the first axis is a minor axis of a cross-section of the self-orienting member, and wherein the second axis is a major axis of the cross-section of the self-orienting member.

Example 5: the catheter of any of examples 2-4, wherein the self-orienting member comprises a hypotube and a spline extending from a distal end of the hypotube.

Example 6: the catheter of example 5, wherein the spline is welded to the distal end of the hypotube.

Example 7: the catheter of any of examples 5 or 6, wherein the spline has a curved cross-section.

Example 8: the catheter of any of examples 5 or 6, wherein the spline has a rectangular cross-section.

Example 9: the catheter of any of examples 5-8, wherein at least one of the hypotube and the spline comprises a metal alloy.

Example 10: the catheter of any of examples 1-9, further comprising an inner tube disposed within the catheter lumen, the inner tube defining an inner lumen in fluid communication with the expandable member.

Example 11: the catheter of any of examples 1-10, wherein a distal portion of the injection tube defines the injection port.

Example 12: the catheter of any of examples 1-11, wherein the preferentially curving configures the catheter body to self-orient the injection port in a predetermined orientation relative to an anatomical feature of the patient when the catheter is positioned in the vasculature of the patient.

Example 13: the catheter of example 12, wherein the anatomical feature comprises a renal vein, and wherein in the predetermined orientation, the injection port faces away from the renal vein.

Example 14: the catheter of any of examples 12 or 13, wherein the catheter body is configured to self-orient the injection port in the predetermined orientation as the catheter extends around the curvature in the vasculature of the patient.

Example 15: the catheter of any of examples 1-14, wherein the first direction corresponds to a first plane orthogonal to a longitudinal axis of the catheter, and wherein the second direction corresponds to a second plane parallel to the longitudinal axis.

Example 16: the catheter of any of examples 1-15, wherein the catheter is configured to deliver a therapy to tissue of the patient by at least introducing a therapeutic agent from the injection port and into the tissue.

Example 17: the catheter of any of examples 1-16, wherein the catheter body comprises: a self-orienting member configured to cause the preferential curvature, the self-orienting member comprising: a first polymer disposed around a first portion of a cross-section of the self-orienting member; and a second polymer disposed around a second portion of the cross-section of the self-orienting member, wherein the second polymer is more rigid than the first polymer.

Example 18: the catheter of example 17, wherein the first portion of the cross-section comprises a majority of the cross-section of the self-orienting member.

Example 19: the catheter of any of examples 1-16, wherein the catheter body comprises a braid comprising a first plurality of strands comprising a first material and a second plurality of strands comprising a second material, and wherein the second material is more rigid than the first material.

Example 20: the catheter of example 19, wherein the first material comprises a polymer.

Example 21: the catheter of any of examples 19 or 20, wherein the second material comprises a metal alloy.

Example 22: the catheter of any of examples 1-21, further comprising a guidewire

tube disposed within the catheter lumen, the guidewire tube defining a guidewire lumen configured to receive a guide member.

Example 23: the catheter of any of examples 1-22, wherein the self-orienting member has a length of between 10 centimeters and 40 centimeters.

Example 24: the catheter of any of examples 1-23, further comprising a radiopaque marker disposed on a distal portion of the injection tube.

Example 25: a catheter comprising: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to self-orient the injection port in a pre-determined orientation relative to an anatomical feature of a patient when the catheter is positioned in vasculature of the patient.

Example 26: the catheter of example 25, wherein the catheter body comprises a self-orienting member configured to cause the self-orientation of the injection port.

Example 27: the catheter of example 26, wherein the self-orienting member is configured to deform relative to a first axis of the self-orienting member, and wherein the self-orienting member is configured to maintain rigidity relative to a second axis of the self-orienting member, the second axis being orthogonal to the first axis.

Example 28: the catheter of example 27, wherein the first axis is a minor axis of a cross-section of the self-orienting member, and wherein the second axis is a major axis of the cross-section of the self-orienting member.

Example 29: the catheter of any of examples 26-28, wherein the self-orienting member comprises a hypotube and a spline extending from a distal end of the hypotube.

Example 30: the catheter of example 29, wherein the spline is welded to the distal end of the hypotube.

Example 31: the catheter of any of examples 29 or 30, wherein the spline has a curved cross-section.

Example 32: the catheter of any of examples 29 or 30, wherein the spline has a rectangular cross-section.

Example 33: the catheter of any of examples 29-32, wherein at least one of the hypotube and the spline comprises a metal alloy.

Example 34: the catheter of any of examples 25-33, further comprising an inner tube disposed within the catheter lumen, the inner tube defining an inner lumen in fluid communication with the expandable member.

Example 35: the catheter of any of examples 25-34, wherein a distal portion of the injection tube defines the injection port.

Example 36: the catheter of any of examples 25-35, wherein the anatomical feature comprises a renal vein, and wherein in the predetermined orientation, the injection port faces away from the renal vein.

Example 37: the catheter of any of examples 25-36, wherein the catheter body is configured to self-orient the injection port in the pre-determined orientation as the catheter navigates around a curvature in the vasculature of the patient.

Example 38: the catheter of any of examples 25-37, wherein the catheter is configured to deliver a therapy to tissue of the patient by at least introducing a therapeutic agent from the injection port of the injection tube and into the tissue.

Example 39: a method of delivering therapy to tissue of a patient, the method comprising: navigating a catheter through vasculature of the patient to a target treatment site, wherein the catheter comprises: a catheter body defining a catheter lumen; an expandable member connected to the catheter body; and an injection tube disposed on an outer surface of the expandable member, the injection tube defining an injection lumen and an injection port in fluid communication with the injection lumen, wherein the catheter body is configured to preferentially curve in a first direction over a second direction when the catheter is positioned in the vasculature; expanding the expandable member of the catheter from a collapsed configuration to an expanded configuration; and delivering therapy to the target treatment site via the injection port.

Example 40: the method of example 39, wherein the catheter body comprises a self-orienting member configured to cause the preferential curvature.

Example 41: the method of example 40, wherein the self-orienting member is configured to deform relative to a first axis of the self-orienting member, and wherein the self-orienting member is configured to maintain rigidity relative to a second axis of the self-orienting member, the second axis being orthogonal to the first axis.

Example 42: the method of example 41, wherein the first axis is a minor axis of a cross-section of the self-orienting member, and wherein the second axis is a major axis of the cross-section of the self-orienting member.

Example 43: the method of any of examples 40-42, wherein the self-orienting member comprises a hypotube and a spline extending from a distal end of the hypotube.

Example 44: the method of example 43, wherein the spline is welded to the distal end of the hypotube.

Example 45: the method of any of examples 43 or 44, wherein the spline has a curved cross-section.

Example 46: the method of any of examples 43 or 44, wherein the spline has a rectangular cross-section.

Example 47: the method of any of examples 43-46, wherein at least one of the hypotube and the spline comprise a metal alloy.

Example 48: the method of any of examples 39-47, wherein the catheter further comprises an inner tube disposed within the catheter lumen, the inner tube defining an inner lumen in fluid communication with the expandable member.

Example 49: the method of example 48, wherein expanding the expandable member comprises delivering a fluid through the inner lumen and into the expandable member.

Example 50: the method of any of examples 39-49, wherein navigating the catheter through the vasculature of the patient comprises causing the catheter to traverse a curvature in the vasculature, wherein the catheter body self-orients the injection port in a predetermined orientation relative to an anatomical feature of the patient when the catheter traverses the curvature in the vasculature.

Example 51: the method of example 50, wherein the anatomical feature comprises a renal vein, and wherein in the predetermined orientation, the injection port faces away from the renal vein.

Example 52: the method of any of examples 50 or 51, wherein navigating the catheter through the vasculature of the patient comprises causing the catheter to traverse a curvature in the vasculature, wherein the catheter body self-orients the injection port in the predetermined orientation when the catheter traverses the curvature in the vasculature.

Example 53: the method of any of examples 39-52, wherein the first direction corresponds to a first plane orthogonal to a longitudinal axis of the catheter, and wherein the second direction corresponds to a second plane parallel to the longitudinal axis.

Example 54: the method of any of examples 39-53, wherein delivering the therapy to the target treatment site via the injection port comprises introducing a therapeutic agent from the injection port of the injection tube and into the tissue.

Example 55: the method of any of examples 39-54, further comprising: collapsing the expandable member from the expanded configuration to the collapsed configuration; navigate the catheter through the vasculature of the patient to a second target treatment site; expanding the expandable member from the collapsed configuration to the expanded configuration; and delivering therapy to the second target treatment site via the injection port.

Example 56: the method of any of examples 29-55, wherein the catheter body comprises a first polymer disposed around a first portion of a cross-section of the self-orienting member and a second polymer disposed around a second portion of the cross-section of the self-orienting polymer, wherein the second polymer is more rigid than the first polymer.

Example 57: the method of example 56, wherein the first portion of the cross-section comprises a majority of the cross-section of the self-orienting member and wherein the second portion comprises a minority portion of the cross-section of the self-orienting member.

Example 58: the method of any of examples 39-57, wherein the catheter body comprises a braid comprising a first plurality of strands comprising a first material and a second plurality of strands comprising a second material, and wherein the second material is more rigid than the first material.

Example 59: the method of example 58, wherein the first material comprises a polymer.

Example 60: the method of any of examples 58 and 59, wherein the second material comprise a metal alloy.

Example 61: the method of any of examples 39-60, further comprising a guidewire tube disposed within the catheter lumen, the guidewire tube defining a guidewire lumen configured to receive a guide member.

Example 62: the method of any of examples 39-61, wherein the self-orienting member has a length of between 10 centimeters and 40 centimeters.

Example 63: the method of any of examples 39-62, wherein the catheter further comprises a radiopaque marker disposed on a distal portion of the injection tube.

Example 64: a catheter comprising: a catheter body; and a neuromodulation element disposed on a distal portion of the catheter body, wherein the catheter body is configured to preferentially curve in a first direction over a second direction when the catheter is positioned in a curvature in vasculature of a patient.

Example 65: the catheter of example 64, wherein the catheter body comprises a self-orienting member configured to cause the preferential curvature.

Example 66: the catheter of example 65, wherein the self-orienting member is configured to deform relative to a first axis of the self-orienting member, and wherein the self-orienting member is configured to maintain rigidity relative to a second axis of the self-orienting member, the second axis being orthogonal to the first axis.

Example 67: the catheter of example 66, wherein the first axis is a minor axis of a cross-section of the self-orienting member, and wherein the second axis is a major axis of the cross-section of the self-orienting member.

Example 68: the catheter of any of examples 65-67, wherein the self-orienting member comprises a hypotube and a spline extending from a distal end of the hypotube.

Example 69: the catheter of example 68, wherein the spline is welded to the distal end of the hypotube.

Example 70: the catheter of any of examples 68 or 69, wherein the spline has a curved cross-section.

Example 71: the catheter of any of examples 69 or 70, wherein the spline has a rectangular cross-section.

Example 72: the catheter of any of examples 69-71, wherein at least one of the hypotube and the spline comprises a metal alloy.

Example 73: the catheter of any of examples 64-72, wherein the neuromodulation element is configured to deliver neuromodulation therapy to an area of influence, and wherein the area of influence extends less than 360 degrees around a circumference of a blood vessel of the patient.

Example 74: the catheter of any of examples 64-73, wherein the preferentially curving configures the catheter body to self-orient a therapy delivery region on the neuromodulation element in a predetermined orientation relative to an anatomical feature of the patient when the catheter is positioned in the vasculature of the patient.

Example 75: the catheter of example 74, wherein the anatomical feature comprises a renal vein, and wherein in the predetermined orientation, the injection port faces away from the renal vein.

Example 76: the catheter of any of examples 74 or 75, wherein the catheter body is configured to self-orient the therapy delivery region in the predetermined orientation as the catheter extends around the curvature in the vasculature of the patient.

Example 77: the catheter of any of examples 74-76, wherein the therapy delivery region on the neuromodulation element extends less than 360 degrees around a circumference of the neuromodulation element.

Example 78: the catheter of any of examples 64-77, wherein the first direction corresponds to a first plane orthogonal to a longitudinal axis of the catheter, and wherein the second direction corresponds to a second plane parallel to the longitudinal axis.

Example 79: the catheter of any of examples 64-78, wherein the catheter is configured to deliver a therapy to tissue of the patient by at least introducing a therapeutic agent from the neuromodulation element into the tissue.

Example 80: the catheter of any of examples 64-78, wherein the neuromodulation element comprises one or more electrodes, and wherein the one or more electrodes is configured to deliver a therapy to tissue of the patient.

Example 81: the catheter of example 80, wherein the one or more electrodes is configured to deliver radiofrequency (RF) energy to the tissue.

Example 82: the catheter of example 80, wherein the one or more electrodes is configured to deliver microwave energy to the tissue.

Example 83: the catheter of example 80, wherein the one or more electrodes is configured to deliver ultrasound energy to the tissue.

Example 84: the catheter of any of examples 64-78, wherein the neuromodulation element comprises an expandable member, and wherein the expandable member is configured to deliver cryogenic energy to tissue of the patient.

Example 85: the catheter of any of examples 64-84, wherein the catheter body comprises: a self-orienting member configured to cause the preferential curvature, the self-orienting member comprising: a first polymer disposed around a first portion of a cross-section of the self-orienting member; and a second polymer disposed around a second portion of the cross-section of the self-orienting member, wherein the second polymer is more rigid than the first polymer.

Example 86: the catheter of example 85, wherein the first portion of the cross-section comprises a majority of the cross-section of the self-orienting member.

Example 87: the catheter of any of examples 64-86, wherein the catheter body comprises a braid comprising a first plurality of strands comprising a first material and a second plurality of strands comprising a second material, and wherein the second material is more rigid than the first material.

Example 88: the catheter of example 87, wherein the first material comprises a polymer.

Example 89: the catheter of any of examples 87 or 88, wherein the second material comprises a metal alloy.

Example 90: the catheter of any of examples 64-89, wherein the catheter body defines a catheter lumen, and wherein a guidewire tube is disposed within the catheter lumen, the guidewire tube defining a guidewire lumen configured to receive a guide member.

Example 91: the catheter of any of examples 64-90, wherein the self-orienting member has a length of between 10 centimeters and 40 centimeters.

Example 92: the catheter of any of examples 64-91, further comprising a radiopaque marker disposed on a distal portion of the catheter body.

1. A catheter comprising:
a catheter body defining a catheter lumen;
an expandable member connected to the catheter body; and
an injection tube disposed on an outer surface of the expandable member, the injection tube defining an injection lumen and an injection port in fluid communication with the injection lumen,
wherein the catheter body is configured to preferentially curve in a first direction over a second direction when the catheter is positioned in a curvature in vasculature of a patient.

2. The catheter of claim 1, wherein the catheter body comprises a self-orienting member configured to cause the preferential curvature, wherein the self-orienting member is configured to deform relative to a first axis of the self-orienting member, and wherein the self-orienting member is configured to maintain rigidity relative to a second axis of the self-orienting member, the second axis being orthogonal to the first axis.

3. The catheter of claim 2, wherein the first axis is a minor axis of a cross-section of the self-orienting member, and wherein the second axis is a major axis of the cross-section of the self-orienting member.

4. The catheter of claim 2, wherein the self-orienting member comprises a hypotube and a spline extending from a distal end of the hypotube.

5. The catheter of claim 4, wherein the spline has a curved cross-section or a rectangular cross-section.

6-7. (canceled)

8. The catheter of claim 1, wherein the preferentially curving configures the catheter body to self-orient the injection port in a predetermined orientation relative to an anatomical feature of the patient when the catheter is positioned in the vasculature of the patient.

9. The catheter of claim 8, wherein the anatomical feature comprises a renal vein, and wherein in the predetermined orientation, the injection port faces away from the renal vein.

10. The catheter of claim 8, wherein the catheter body is configured to self-orient the injection port in the predetermined orientation as the catheter extends around the curvature in the vasculature of the patient.

11. The catheter of claim 1, wherein the first direction corresponds to a first plane orthogonal to a longitudinal axis of the catheter, and wherein the second direction corresponds to a second plane parallel to the longitudinal axis.

12. (canceled)

13. The catheter of claim 1, wherein the catheter body comprises:
a self-orienting member configured to cause the preferential curvature, the self-orienting member comprising:
a first polymer disposed around a first portion of a cross-section of the self-orienting member; and
a second polymer disposed around a second portion of the cross-section of the self-orienting member, wherein the second polymer is more rigid than the first polymer.

14. The catheter of claim 13, wherein the first portion of the cross-section comprises a majority of the cross-section of the self-orienting member.

15. The catheter of claim 1, wherein the catheter body comprises a braid comprising a first plurality of strands comprising a first material and a second plurality of strands comprising a second material, and wherein the second material is more rigid than the first material, and wherein one or more of:
the first material comprises a polymer; or the second material comprises a metal alloy.

16. A method of delivering therapy to tissue of a patient, the method comprising:
navigating a catheter through vasculature of the patient to a target treatment site, wherein the catheter comprises:
a catheter body defining a catheter lumen;
an expandable member connected to the catheter body; and
an injection tube disposed on an outer surface of the expandable member, the injection tube defining an injection lumen and an injection port in fluid communication with the injection lumen,
wherein the catheter body is configured to preferentially curve in a first direction over a second direction when the catheter is positioned in the vasculature;
expanding the expandable member of the catheter from a collapsed configuration to an expanded configuration; and
delivering therapy to the target treatment site via the injection port.

17. The method of claim 16, wherein the catheter body comprises a self-orienting member configured to cause the preferential curvature, and wherein the self-orienting member is configured to deform relative to a minor axis of a cross-section of the self-orienting member, and wherein the self-orienting member is configured to maintain rigidity relative to a major axis of the cross-section of the self-orienting member.

18. The method of claim 17, wherein the self-orienting member comprises a hypotube and a spline extending from a distal end of the hypotube.

19. The method of claim 16, wherein navigating the catheter through the vasculature of the patient comprises causing the catheter to traverse a curvature in the vasculature, wherein the catheter body self-orients the injection port in a predetermined orientation relative to an anatomical feature of the patient when the catheter traverses the curvature in the vasculature.

20. The method of claim 19, wherein navigating the catheter through the vasculature of the patient comprises causing the catheter to traverse a curvature in the vasculature, wherein the catheter body self-orients the injection port in the predetermined orientation when the catheter traverses the curvature in the vasculature.

21. A catheter comprising:
a catheter body, wherein the catheter body comprises a self-orienting member; and
a neuromodulation element disposed on a distal portion of the catheter body,
wherein the self-orienting member is configured to cause the catheter body to preferentially curve in a first direction over a second direction when the catheter is positioned in a curvature in vasculature of a patient.

22. The catheter of claim 21, wherein the self-orienting member is configured to deform relative to a minor axis of a cross-section of the self-orienting member, and wherein the self-orienting member is configured to maintain rigidity relative to a major axis of a cross-section of the self-orienting member, the minor axis being orthogonal to the major axis.

23. The catheter of claim 21, wherein the neuromodulation element is configured to deliver neuromodulation therapy to an area of influence, and wherein the area of influence extends less than 360 degrees around a circumference of a blood vessel of the patient.
